(19) United States
(12) Patent Application Publication
(10) Pub. No.: US 2022/0139565 A1
(43) Pub. Date: May 5, 2022

(54) GROUP-BASED STRATIFIED TRAJECTORY MODELING

(71) Applicant: International Business Machines Corporation, Armonk, NY (US)

(72) Inventors: Toshiya Iwamori, Tokyo (JP); Akira Koseki, Yokohama-shi (JP)

(21) Appl. No.: 17/089,065

(22) Filed: Nov. 4, 2020

(51) Int. Cl. G16H 50/70; G16H 10/60; G16H 50/20; G16H 50/30

(52) U.S. Cl. CPC G16H 50/70; G16H 50/30; G16H 50/20; G16H 10/60

(57) ABSTRACT

A computer-implemented method, a computer program product, and a computer system for tracking progression of chronic conditions. A computer acquires trajectories of estimated glomerular filtration rates of respective patients. The computer determines a number of trajectory parts in each of the trajectories. The computer generates, in each of response vectors of the trajectories, subsets corresponding to respective ones of the trajectory parts. The computer replaces responses in the response vectors with the subsets. The computer determine, in the respective ones of the trajectory parts, numbers of patient groups. The computer calculates probabilities of the respective patients belonging to respective ones of the patient groups, based on the subsets. The computer clusters the respective patients into the respective ones of the patient groups, based on the probabilities. Information of clustering the patient groups is used to identify risk groups for renal functions.

### B ACKGROUND

The present invention relates generally to tracking the progression of chronic conditions using trajectory modeling, and more particularly to tracking the progression of chronic conditions using group-based stratified trajectory modeling.

Chronic kidney disease (CKD) is associated with a high risk of mortality and morbidity. The widespread use of electronic health record systems (EHRs) has led to advancements in disease progression modeling. EHRs are essential for monitoring and understanding the progression of CKD in order to provide early-stage treatment and prevent further progression of the disease. Recent studies have focused on the rapid decline (RD) in glomerular filtration rate (GFR) in CKD due to its potential association with end-stage kidney disease and cardiovascular disease. Moreover, there has been increasing interests in trajectories of disease progression for chronic diseases.

Group-based trajectory modeling (GBTM) has been used to track the progression of chronic conditions by stratifying patients into groups and detecting risk groups to support decisions regarding treatment and medication. In some cases, GBTM is insufficient for analyzing trajectories. For example, although there are two patients with similar initial estimated glomerular filtration rates (eGFRs) at the start of RD, one patient becomes RD in several months because of the sharply decreasing of renal function and another patient takes time until becoming RD. The fall of eGFR in RD depends on each patient. For clinical decision making, it is essential to identify risk factors and prognoses depending on how it descends with similar initial eGFRs. However, GBTM has the problem for a clustering. Because GBTM depends on initial value of trajectories when clustering trajectories, GBTM cannot divide trajectories into initial eGFRs of trajectories and shapes of trajectories independently.

Additionally, although GBTM uses a cubic expression to represent eGFR trajectories, GBTM cannot adequately capture the characteristics of the eGFR. As a patient's condition improves or declines, the trajectory continues to increase or decrease. A cubic expression cannot represent such a trajectory. However, it is difficult to use a higher dimensional expression due to it takes too much computational cost.

### SUMMARY

In one aspect, a computer-implemented method for tracking progression of chronic conditions is provided. The computer-implemented method includes acquiring, from an electronic health record system, trajectories of estimated glomerular filtration rates of respective patients. The computer-implemented method further includes determining, in each of the trajectories, a predetermined number of trajectory parts. The computer-implemented method further includes generating, in each of response vectors of respective ones of the trajectories, subsets corresponding to respective ones of the trajectory parts. The computer-implemented method further includes replacing responses in the response vectors with the subsets. The computer-implemented method further includes determine, in the respective ones of the trajectory parts, numbers of patient groups. The computer-implemented method further includes calculating, based on the subsets, probabilities of the respective patients belonging to respective ones of the patient groups. The computer-implemented method further includes clustering the respective patients into the respective ones of the patient groups, based on the probabilities, where information of clustering the patient groups is used to identify risk groups for renal functions of the respective patients.

In one embodiment of the computer-implemented method, the number of the trajectory parts is equal to 2. In such an embodiment, computer-implemented method includes: determining that each of the trajectories is to be divided into an initial part and a trajectory-shape part; generating, in each of the response vectors of respective ones of the trajectories, a first subset corresponding to the initial part; generating, in each of the response vectors of the respective ones of the trajectories, a second subset corresponding to the trajectory-shape part; and calculating, based on the first subset and the second subset, the probabilities of the respective patients belonging to the respective ones of the patient groups. In the embodiment, the first subset includes an initial response at an initial time, and the second subset is obtained by subtracting an initial response at the initial time from respective ones of the responses after the initial time.

In another aspect, a computer program product for tracking progression of chronic conditions is provided. The computer program product comprises a computer readable storage medium having program instructions embodied therewith, and the program instructions are executable by one or more processors. The program instructions are executable to: acquire, from an electronic health record system, trajectories of estimated glomerular filtration rates of respective patients; determine, in each of the trajectories, a number of trajectory parts; generate, in each of response vectors of respective ones of the trajectories, subsets corresponding to respective ones of the trajectory parts; replace responses in the response vectors with the subsets; determine, in the respective ones of the trajectory parts, numbers of patient groups; calculate, based on the subsets, probabilities of the respective patients belonging to respective ones of the patient groups; cluster the respective patients into the respective ones of the patient groups, based on the probabilities, where information of clustering the patient groups is used to identify risk groups for renal functions of the respective patients.

In one embodiment of the computer program product, the number of the trajectory parts is equal to 2. In this embodiment, the program instructions are executable to: determine that each of the trajectories is to be divided into an initial part and a trajectory-shape part; generate, in each of the response vectors of respective ones of the trajectories, a first subset corresponding to the initial part; generate, in each of the response vectors of the respective ones of the trajectories, a second subset corresponding to the trajectory-shape part; and calculate, based on the first subset and the second subset, the probabilities of the respective patients belonging to the respective ones of the patient groups. In such an embodiment of computer program product, the first subset includes an initial response at an initial time and the second subset is obtained by subtracting an initial response at the initial time from respective ones of the responses after the initial time.

In yet another aspect, a computer system for tracking progression of chronic conditions is provided. The computer system comprises one or more processors, one or more computer readable tangible storage devices, and program instructions stored on at least one of the one or more computer readable tangible storage devices for execution by at least one of the one or more processors. The program instructions are executable to acquire, from an electronic health record system, trajectories of estimated glomerular filtration rates of respective patients. The program instructions are further executable to determine, in each of the trajectories, a number of trajectory parts. The program instructions are further executable to generate, in each of response vectors of respective ones of the trajectories, subsets corresponding to respective ones of the trajectory parts. The program instructions are further executable to replace responses in the response vectors with the subsets. The program instructions are further executable to determine, in the respective ones of the trajectory parts, numbers of patient groups. The program instructions are further executable to calculate, based on the subsets, probabilities of the respective patients belonging to respective ones of the patient group. The program instructions are further executable to cluster the respective patients into the respective ones of the patient groups, based on the probabilities. Information of clustering the patient groups is used to identify risk groups for renal functions of the respective patients.

In one embodiment of the computer system, the number of the trajectory parts is equal to 2. In the embodiment of the computer system, the program instructions are executable to divide each of the trajectories into an initial part and a trajectory-shape part; determine that each of the trajectories is to be divided into an initial part and a trajectory-shape part; the program instructions are further executable to generate, in each of the response vectors of respective ones of the trajectories, a first subset corresponding to the initial part; the program instructions are further executable to generate, in each of the response vectors of the respective ones of the trajectories, a second subset corresponding to the trajectory-shape part; and the program instructions are further executable to calculate, based on the first subset and the second subset, the probabilities of the respective patients belonging to the respective ones of the patient groups. In the embodiment of the computer system, the first subset includes an initial response at an initial time, while the second subset is obtained by subtracting an initial response at the initial time from respective ones of the responses after the initial time.

### BRIEF DESCRIPTION OF THE SEVERAL VIEWS OF THE DRAWINGS

FIG. 1 presents a flowchart showing operational steps of tracking the progression of chronic conditions using group-based stratified trajectory modeling, in accordance with one embodiment of the present invention.

FIG. 2 presents a flowchart showing operational steps of tracking the progression of chronic conditions using group-based stratified trajectory modeling, in accordance with another embodiment of the present invention.

FIG. 3 is a diagram illustrating components of a computing device or server, in accordance with one embodiment of the present invention.

FIG. 4 depicts a cloud computing environment, in accordance with one embodiment of the present invention.

FIG. 5 depicts abstraction model layers in a cloud computing environment, in accordance with one embodiment of the present invention.

### DETAILED DESCRIPTION

Embodiments of the present invention propose a new clustering algorithm, group-based stratified trajectory modeling (GBSTM). GBSTM independently stratifies the value of initial estimated glomerular filtration rate (eGFR) and the shape of the trajectory, and use more suitable expression to capture trajectory characteristics. GBSTM stratifies initial eGFR and then separates the trajectories on the basis of the initial eGFR of each group of patients. GBSTM estimates the expression parameters using each point on the trajectory to represent a complex curve that cannot be represented by a cubic expression. The proposed approach uses a linear expression which has a relatively low computational cost.

Group-based stratified trajectory modeling (GBSTM) extends group-based trajectory modeling (GBTM) that uses past cell states weighted by attention and discounted by time decay function. GBSTM replaces response vector y to separate y into L groups by functions for separating y to several parts. Then, y is replaced with clusters of several parts for y. Let y be the response variable for the eGFR. Then, y=={y₁, . . . , y_{T}} is a vector of length T. The mathematical expressions for this are as follows:

\tilde{y}ⱼ=gⱼ(y), for j=1,2, . . . , L (1)

y={ [symbol] , . . . , [symbol] } (2)

where L is the total number of separated parts of the response vector y, g ( )is a function for generating the subsets { [symbol] , . . . , [symbol] } from the response vector. Then, group-based stratified trajectory modeling (GBSTM) uses a density function as follows:

$$f ( y ) = {\sum }_{{k}_{1} = 1}^{{K}_{1}} … {\sum }_{{k}_{L} = 1}^{{K}_{L}} {p}_{{k}_{1}} {p}_{{k}_{1} , {k}_{2}} … {p}_{{k}_{1} , … , {k}_{j}} … {p}_{{k}_{1} , … , {k}_{L}} {\prod }_{j = 1}^{L} {\prod }_{d = 1}^{{D}_{j}} ( \vert {C}_{{k}_{1}} = {k}_{1} , … , {C}_{{k}_{j}} = {k}_{j} ) \qquad ( 3 )$$

K₁, . . . , K_{L} denote total numbers of groups for respective separated groups, Dⱼ denotes the number of the dimension for the j-th part of y, Cₖ_{ⱼ} is the class which \tilde{y}ⱼ belongs to, pₖ_{ⱼ} values are the probabilities of belonging to groups k₁, k₂, . . . , and k₁, respectively. θ is a kⱼ-dimensional vector of class coefficients. If each pₖ_{₁}_{, . . . , k}_{ⱼ} is independent, [symbol] ⱼ (equation (5) is used. [symbol] ( [symbol] |Cₖ_{₁}=k₁, . . . , Cₖ_{ⱼ}=kⱼ) is the conditional density function of the observed data given classes k₁, k₂, . . . , and kⱼ. N is a normal distribution, σ_{j,d}² is a variance, and μ_{j,d,k}_{₁}_{, . . . , k}_{ⱼ} ∈ R represents elements of the mean vector and can be determined based on β_{d,k}_{₁}_{, . . . , k}_{ⱼ}. β_{d,k}_{₁}_{, . . . , k}_{ⱼ} ∈ R is a parameter for a trajectory shape and is estimated by using the Bayesian model, φ is a function defined by β_{d,k}_{₁}_{, . . . ,k}_{ⱼ}.

Let L=2 , group-based stratified trajectory modeling (GBSTM) replaces response vector y to divide y into the initial eGFR part ( [symbol] ) and the trajectory shape part ( [symbol] ) for clustering based on each characteristic. GBSTM subtracts the initial eGFR y₁ from the response vector except for the initial eGFR to obtain [symbol] . The initial eGFRs is ignored from the second point when clustering to separate trajectory shapes. Then, y is replaced by [symbol] and [symbol] . This is mathematically described as follows:

[symbol]

=g₁(y)={y₁} (8)

[symbol]

=g₂(y)={y₂−y₁, . . . , y₁} (9)

y={ [symbol] } (10)

where the total number of separated parts of the response vector y is 2, g₁( ) and g₂( ) are functions for generating the subsets { [symbol] } from the response vector.

After replacing response vector y, GBSTM uses a density function and y is modeled as follows.

$$\begin{matrix}{p}_{{k}_{1} , … {k}_{j}} = \frac{{e}^{{θ}_{{k}_{1} , … , {k}_{j}}}}{{\sum }_{{l}_{j} = 1}^{{K}_{j}} {e}^{{θ}_{{k}_{1} , … {l}_{j}}}} & ( 4 ) \\ = \frac{{e}^{{θ}_{{k}_{j}}}}{{\sum }_{{l}_{j} = 1}^{{K}_{j}} {e}^{{θ}_{{k}_{j}}}} & ( 5 ) \\ \vert {C}_{{k}_{1}} = {k}_{1} , … , {C}_{{k}_{j}} = {k}_{j} ∼ N ( {μ}_{j , d , {k}_{1} , … , {k}_{j}} , {σ}_{j , d}^{2} ) & ( 6 ) \\ {μ}_{j , d , {k}_{1} , … , {k}_{j}} = {φ}_{{k}_{j}} ( {β}_{d , {k}_{1} , … , {k}_{j}} ) & ( 7 )\end{matrix}$$

K₁ denotes the total number of groups for the initial eGFR, K₂ denotes the total number of groups for the trajectory shapes, Dⱼ is the number of the dimension for the j-th part of y, Cₖ_{₁} and Cₖ_{₂} are the classes which the trajectory belongs to. pₖ_{₁} is the probability of belonging to class k₁, while pₖ_{₂}is the probability of belonging to class k₂. θ is a k-dimensional vector of class coefficients. [symbol] ( [symbol] |Cₖ_{₁}=k₁, Cₖ_{₂}=k₂) is the conditional density function of the observed data given classes k₁ and k₂. N is a normal distribution. σ_{1,d}² and σ_{2,d}² are variances. μ_{1,k}_{₁} and μ_{2,d,k}_{₁}_{,k}_{₂} ∈ R represent elements of the mean vector and can be determined based βₖ_{₁} and β_{d,k}_{₁}_{,k}_{₂}. βₖ_{₁} and β_{d,k}_{₁}_{,k}_{₂} ∈ R are parameters of trajectory shapes and are estimated by using the Bayesian model. t_{d} is a time stamp at [symbol] .

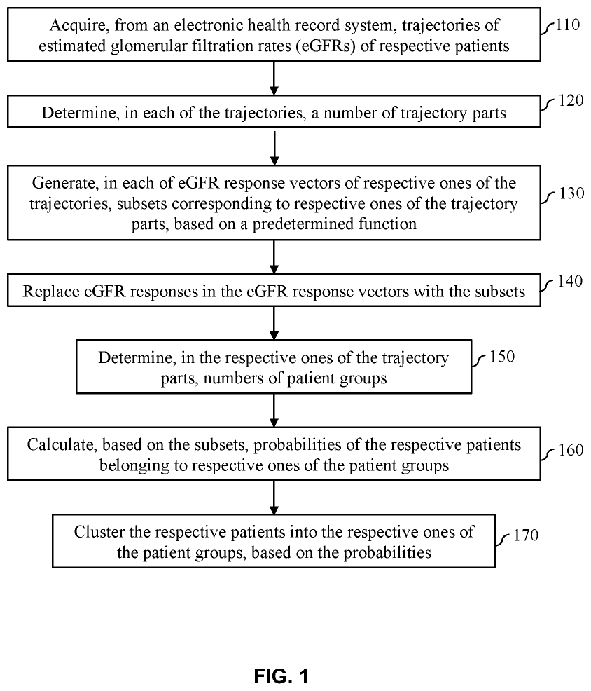

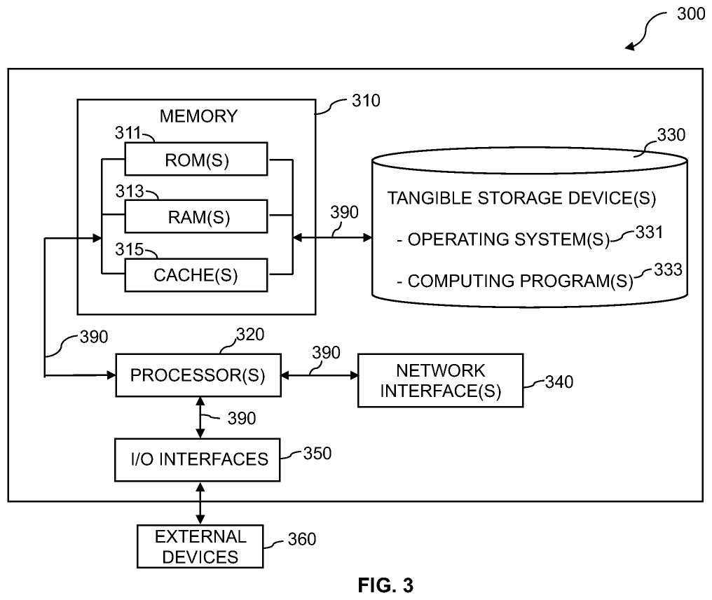

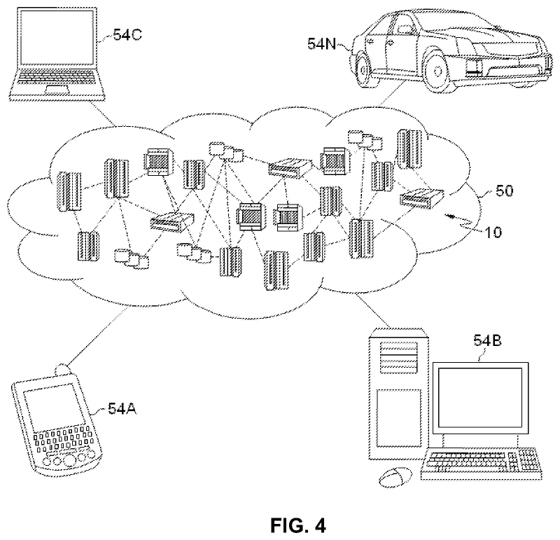

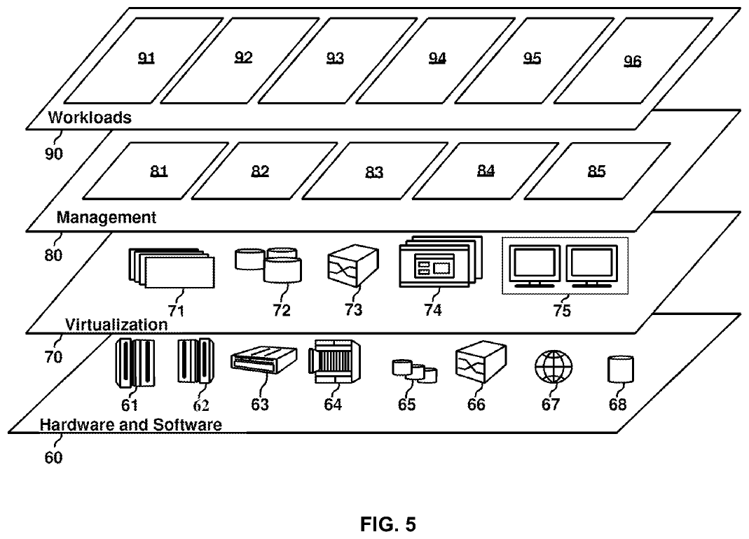

FIG. 1 presents a flowchart showing operational steps of tracking the progression of chronic conditions using group-based stratified trajectory modeling, in accordance with one embodiment of the present invention. The operational steps are implemented by a computing device or sever. A computing device or server is described in more detail in later paragraphs with reference to FIG. 3. In another embodiment, the operational steps may be implemented on a virtual machine or another virtualization implementation being run on one or more computing devices or servers. In yet another embodiment, the operational steps may be implemented by a computing device or sever in a cloud computing environment. The cloud computing environment is described in later paragraphs with reference to FIG. 4 and FIG. 5.

At step 110, a computing device or server acquires, from an electronic health record system, trajectories of estimated glomerular filtration rates (eGFRs) of respective patients. For a respective patient, the computer device or server acquires an eGFR response vector y={y₁, . . . , y_{T}}. The eGFR response vector includes eGFR responses at times (t=1, . . . , t=T), and the eGFR response vector represents an eGFR trajectory of the respective patient. The electronic health record system is an electronic version of a patients medical history. In acquiring, processing, storing, and using the data from the electronic health record system, laws are observed and privacy of the respective patients is protected.

At step 120, the computing device or server determines, in each of the trajectories, a number of trajectory parts. Let the total number of the trajectory parts is L. At step 130, in each of eGFR response vectors of respective ones of the trajectories, the computing device or server generates subsets corresponding to respective ones of the trajectory parts. Generating the subsets is based on predetermined functions for the respective ones of the trajectory parts. For a respective patient, an eGFR response vector is y={y₁, . . . , y_{T}}; the computing device or server divides the vector y into multiple subsets and the total number of the subsets is L. Each subset corresponds one trajectory part. As described in equations (1) and (2), the subsets are denoted as [symbol] , . . . , [symbol] . Each subset is generated based on a function, as described in equation (1).

At step 140, the computing device or server replaces eGFR responses in the eGFR response vectors with the subsets. For a respective trajectory of a respective patient, eGFR responses y₁, . . . , y_{T} in an eGFR response vector y is replaced by [symbol] , . . . , [symbol] ; thus, the eGFR response vector becomes y={ [symbol] , . . . , [symbol] }, as described in equation (2).

Now, the trajectories for the respective patients have been divided into L trajectory parts; corresponding to each trajectory part, a subset is determined. Next, for each of the trajectory parts, the respective patients will be clustered into patients groups.

At step 150, the computing device or server determines, in the respective ones of the trajectory parts, numbers of patient groups. At step 160, the computing device or server calculates, based on the subsets, probabilities of the respective patients belonging to respective ones of the patient groups. Estimation of the probabilities can be based on equation (4) or (5). At step 170, the computing device or server clusters the respective patients into the respective ones of the patient groups, based on the probabilities calculated at step 160. The information of clustering the patient groups is used to identify risk groups for renal functions of the respective patients.

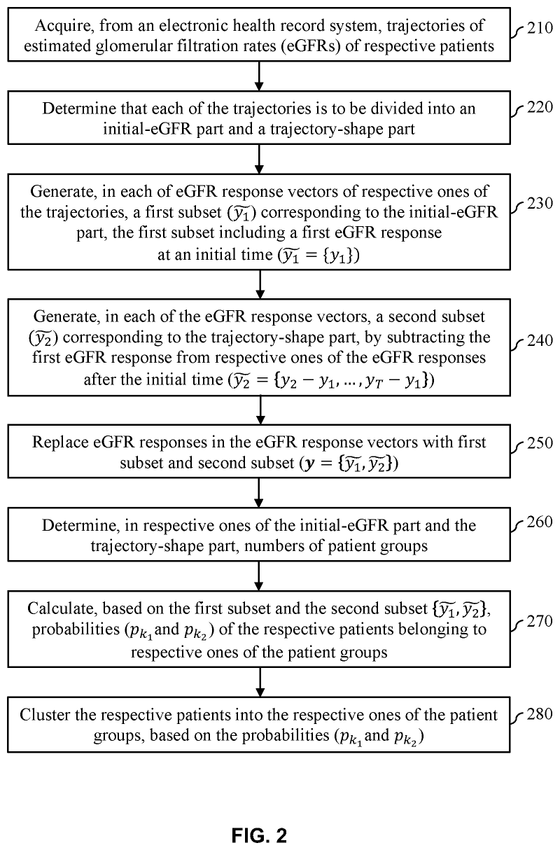

FIG. 2 presents a flowchart showing operational steps of tracking the progression of chronic conditions using group-based stratified trajectory modeling, in accordance with another embodiment of the present invention. In this embodiment, the total number of separated parts of the response vector y is equal to 2, i.e., L=2. The operational steps are implemented by a computing device or sever. A computing device or server is described in more detail in later paragraphs with reference to FIG. 3. In another embodiment, the operational steps may be implemented on a virtual machine or another virtualization implementation being run on one or more computing devices or servers. In yet another embodiment, the operational steps may be implemented by a computing device or sever in a cloud computing environment. The cloud computing environment is described in later paragraphs with reference to FIG. 4 and FIG. 5.

At step 210, a computing device or server acquires, from an electronic health record system, trajectories of estimated glomerular filtration rates (eGFRs) of respective patients. For a respective one of the patients, the computer device or server acquires an eGFR response vector y={y₁, . . . , y_{T}}, which represents an eGFR trajectory of a respective patient. The eGFR response vector includes eGFR responses (y₁, . . . , y_{T}) at times (t=1, . . . , t=T). In acquiring, processing, storing, and using the data from the electronic health record system, laws are observed and privacy of the respective patients is protected.

At step 220, the computing device or server determines that each of the trajectories is to be divided into an initial-eGFR part and a trajectory-shape part. The total number of separated parts of the response vector y is equal to 2. Each of the trajectories is divided into 2 trajectory parts (the initial-eGFR part and the trajectory-shape part).

At step 230, the computing device or server generates, in each of eGFR response vectors of respective ones of the trajectories, a first subset ( [symbol] ) corresponding to the initial-eGFR part. The first subset includes a first eGFR response (y₁) at an initial time (t=1). As described in equation (8), the first subset [symbol] includes only the initial eGFR response y₁, and the function for generating the first subset [symbol] from the response vector y is: g₁(y)={y₁}.

At step 240, the computing device or server generates, in each of the eGFR response vectors, a second subset ( [symbol] ) corresponding to the trajectory-shape part. The second subset [symbol] is generated by subtracting the first eGFR response y₁ from respective ones of the eGFR responses (y₂, . . . , y_{T}) after the initial time. As described in equation (9), the second subset is: {y₂−y₁, . . . , y_{T}−y₁}. The function for generating the second subset [symbol] from the response vector y is: g₂ (y)={y₂−y₁, . . . , y_{T}−y₁}.

At step 250, the computing device or server replaces eGFR responses in the eGFR response vectors with first subset and second subset. The first subset is corresponding to the initial-eGFR part, while the second subset is corresponding to trajectory-shape part. Thus, y is equal to { [symbol] }, as described in equation (10).

At step 260. the computing device or server determines, in respective ones of the initial-eGFR part and the trajectory-shape part, numbers of patient groups. At step 270, the computing device or server calculates, based on the first subset ( [symbol] ) and the second subset ( [symbol] ), probabilities (pₖ_{₁} and pₖ_{₂}) of the respective patients belonging to respective ones of the patient groups. Estimation of the probabilities (pₖ_{₁}and pₖ_{₂}) can be based on equation (12) and (13), respectively. At step 280, the computing device or server clusters the respective patients into the respective ones of the patient groups, based on the probabilities (pₖ_{₁} and pₖ_{₂}). The information of clustering the patient groups is used to identify risk groups for renal functions of the respective patients.

FIG. 3 is a diagram illustrating components of a computing device or server, in accordance with one embodiment of the present invention. It should be appreciated that FIG. 3 provides only an illustration of one implementation and does not imply any limitations with regard to the environment in which different embodiments may be implemented.

Referring to FIG. 3, computing device or server 300 includes processor(s) 320, memory 310, and tangible storage device(s) 330. In FIG. 3, communications among the above-mentioned components of computing device or server 300 are denoted by numeral 390. Memory 310 includes ROM(s) (Read Only Memory) 311, RAM(s) (Random Access Memory) 313, and cache(s) 315. One or more operating systems 331 and one or more computer programs 333 reside on one or more computer readable tangible storage device(s) 330.

Computing device or server 300 further includes I/O interface(s) 350. I/O interface(s) 350 allows for input and output of data with external device(s) 360 that may be connected to computing device or server 300. Computing device or server 300 further includes network interface(s) 340 for communications between computing device or server 300 and a computer network.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the C programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as Follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as Follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as Follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

Referring now to FIG. 4, illustrative cloud computing environment 50 is depicted. As shown, cloud computing environment 50 includes one or more cloud computing nodes 10 with which local computing devices are used by cloud consumers, such as mobile device 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N are intended to be illustrative only and that computing nodes 10 and cloud computing environment 50 can communicate with any type of computerized device over any type of network and/or network addressable connection (e.g., using a web browser).

Referring now to FIG. 5, a set of functional abstraction layers provided by cloud computing environment 50 (FIG. 4) is shown. It should be understood in advance that the components, layers, and functions shown in FIG. 5 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and function 96. Function 96 is the functionality of tracking the progression of chronic conditions using group-based stratified trajectory modeling.

What is claimed is:

1. A computer-implemented method for tracking progression of chronic conditions, the method comprising:
acquiring, from an electronic health record system, trajectories of estimated glomerular filtration rates of respective patients;
determining, in each of the trajectories, a number of trajectory parts;
generating, in each of response vectors of respective ones of the trajectories, subsets corresponding to respective ones of the trajectory parts;
replacing responses in the response vectors with the subsets;
determining, in the respective ones of the trajectory parts, numbers of patient groups;
calculating, based on the subsets, probabilities of the respective patients belonging to respective ones of the patient groups;
clustering, based on the probabilities, the respective patients into the respective ones of the patient groups; and
wherein information of clustering the patient groups is used to identify risk groups for renal functions of the respective patients.

2. The computer-implemented method of claim 1, wherein a response vector includes response values of the estimated glomerular filtration rates at different times for a patient.

3. The computer-implemented method of claim 1, wherein the number of the trajectory parts is equal to 2.

4. The computer-implemented method of claim 3, further comprising:
determining that each of the trajectories is to be divided into an initial part and a trajectory-shape part;
generating, in each of the response vectors of respective ones of the trajectories, a first subset corresponding to the initial part;
generating, in each of the response vectors of the respective ones of the trajectories, a second subset corresponding to the trajectory-shape part; and
calculating, based on the first subset and the second subset, the probabilities of the respective patients belonging to the respective ones of the patient groups.

5. The computer-implemented method of claim 4, wherein the first subset includes an initial response at an initial time.

6. The computer-implemented method of claim 4, wherein the second subset is obtained by subtracting an initial response at an initial time from respective ones of the responses after the initial time.

7. The computer-implemented method of claim 1, wherein generating the subsets is based on predetermined functions for the respective ones of the trajectory parts.

8. A computer program product for tracking progression of chronic conditions, the computer program product comprising a computer readable storage medium having program instructions embodied therewith, the program instructions executable by one or more processors, the program instructions executable to:
acquire, from an electronic health record system, trajectories of estimated glomerular filtration rates of respective patients;
determine, in each of the trajectories, a number of trajectory parts;
generate, in each of response vectors of respective ones of the trajectories, subsets corresponding to respective ones of the trajectory parts;
replace responses in the response vectors with the subsets;
determine, in the respective ones of the trajectory parts, numbers of patient groups;
calculate, based on the subsets, probabilities of the respective patients belonging to respective ones of the patient groups;
cluster, based on the probabilities, the respective patients into the respective ones of the patient groups; and
wherein information of clustering the patient groups is used to identify risk groups for renal functions of the respective patients.

9. The computer program product of claim 8, wherein a response vector includes response values of the estimated glomerular filtration rates at different times for a patient.

10. The computer program product of claim 8, wherein the number of the trajectory parts is equal to 2.

11. The computer program product of claim 10, further comprising the program instructions executable to:
determine that each of the trajectories is to be divided into an initial part and a trajectory-shape part;
generate, in each of the response vectors of respective ones of the trajectories, a first subset corresponding to the initial part;
generate, in each of the response vectors of the respective ones of the trajectories, a second subset corresponding to the trajectory-shape part; and
calculate, based on the first subset and the second subset, the probabilities of the respective patients belonging to the respective ones of the patient groups.

12. The computer program product of claim 11, wherein the first subset includes an initial response at an initial time.

13. The computer program product of claim 11, wherein the second subset is obtained by subtracting an initial response at an initial time from respective ones of the responses after the initial time.

14. The computer program product of claim 8, wherein generating the subsets is based on predetermined functions for the respective ones of the trajectory parts.

15. A computer system for tracking progression of chronic conditions, the computer system comprising one or more processors, one or more computer readable tangible storage devices, and program instructions stored on at least one of the one or more computer readable tangible storage devices for execution by at least one of the one or more processors, the program instructions executable to:
acquire, from an electronic health record system, trajectories of estimated glomerular filtration rates of respective patients;
determine, in each of the trajectories, a number of trajectory parts;
generate, in each of response vectors of respective ones of the trajectories, subsets corresponding to respective ones of the trajectory parts;
replace responses in the response vectors with the subsets;
determine, in the respective ones of the trajectory parts, numbers of patient groups;
calculate, based on the subsets, probabilities of the respective patients belonging to respective ones of the patient groups;
cluster, based on the probabilities, the respective patients into the respective ones of the patient groups; and
wherein information of clustering the patient groups is used to identify risk groups for renal functions of the respective patients.

16. The computer system of claim 15, wherein a response vector includes response values of the estimated glomerular filtration rates at different times for a patient.

17. The computer system of claim 15, wherein the number of the trajectory parts is equal to 2.

18. The computer system of claim 17, further comprising the program instructions executable to:
determine that each of the trajectories is to be divided into an initial part and a trajectory-shape part;
generate, in each of the response vectors of respective ones of the trajectories, a first subset corresponding to the initial part;
generate, in each of the response vectors of the respective ones of the trajectories, a second subset corresponding to the trajectory-shape part; and
calculate, based on the first subset and the second subset, the probabilities of the respective patients belonging to the respective ones of the patient groups.

19. The computer system of claim 18, wherein the first subset includes an initial response at an initial time.

20. The computer system of claim 18, wherein the second subset is obtained by subtracting an initial response at an initial time from respective ones of the responses after the initial time.
